(19) United States
(12) Patent Application Publication
(10) Pub. No.: US 2009/0170200 A1
(43) Pub. Date: Jul. 2, 2009

(54) STEM CELL MEDIUM

(75) Inventors: Ching-Hua Yeh, Pingtung County (TW); Gwo-Jaw Wang, Kaohsiung City (TW); Mei-Ling Ho, Kaohsiung City (TW); Je-Ken Chang, Kaohsiung City (TW); Chung-Hwan Chen, Kaohsiung City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: KAOHSIUNG MEDICAL UNIVERSITY, Kaohsiung City (TW)

(21) Appl. No.: 12/106,453

(22) Filed: Apr. 21, 2008

(30) Foreign Application Priority Data
Dec. 31, 2007 (TW) 96151437

(51) Int. Cl. C12N 5/06

(52) U.S. Cl. 435/384; 435/405; 435/406

(57) ABSTRACT

A medium for culturing stem cell. The stem cell medium of the invention comprises a fetal bovine serum, one or plurality of amino acid, one or plurality of vitamin, one or plurality of growth factor, one or plurality of inorganic salt, one or plurality of antioxidant, wherein the stem cell medium has a calcium concentration of less than about 1.8 mM, and the fetal bovine serum is present in an amount of less than about 10% by volume of the medium. The stem cell medium of the invention can maintain the proliferative and self-renewal capacity of the stem cells and keep stem cells at a steady stage.

### BACKGROUND OF THE INVENTION

1. Field of the Invention

The present invention relates to a medium composition, and in particular relates to a medium composition for culturing stem cells and maintaining stem cell properties.

2. Description of the Related Art

Stem cells are cells found in all multi-cellular organisms. They retain the ability to renew themselves through mitotic cell division and can differentiate into a diverse range of specialized cell types. Thus, a stem cell can be regarded as a repair system to supplementing the cells for a biological subject. Medical researchers believe that stem cells (regenerative medicine) provide a potential to change the diseases treatment for repairing specific tissues or organs.

Bone marrow is soft blood-forming living tissue that fills most bone cavities and contains fat, immature and mature blood cells, hematopoietic stem cells and non hematopoietic stem cells, from all red and white blood cells, and platelets. Non hematopoietic stem cells are located at bone marrow stroma. Bone marrow stroma cells also contains mesenchymal stem cells, whereby the stem cells generate bone, cartilage, fat, fibrous connective tissue, and the reticular network. hBMSC (Human bone marrow stem cells) belongs to adult stem cells, and like all stem cells, share at least two characteristics. First, they can make identical copies of themselves for long periods of time; this ability to proliferate is referred to as long-term self-renewal. Second, they can give rise to mature cell types that have characteristic morphologies (shapes) and specialized functions.

Human mesenchymal stem cells are pluripotent bone marrow cells that can be expanded ex vivo and differentiated into several mesodermal lineages, such as cartilage, bone, and fat, it has the ability to develop from the three germ layers (mesoderm, endoderm, and ectoderm) from which all the cells of the body arise. They usually divide to generate progenitor or precursor cells, which then differentiate or develop into “mature” cell types that have characteristic shapes and specialized functions.

However, the frequency of hBMSC on human bone marrow stroma cells is not high, maintenance and expansion of these cells in culture condition are difficult, and these cells on culture condition are easy triggered for differentiation or die. Accordingly, the formula for efficient culture and growth of adult HBMSC is useful and necessary for people to yield good and huge hBMSC. Thus, a good condition is needed to amplify stem cells and keep stem cells at a steady stage, which would be easier for triggering differentiation.

### BRIEF SUMMARY OF INVENTION

The invention provides a medium of culturing stem cells, comprising a fetal bovine serum, one or more amino acid, one or more vitamin, one or more growth factor, one or more inorganic ion salt, one or more antioxidant agent, wherein the medium has a calcium concentration of less than 1.8 mM, and the fetal bovine serum is present in an amount of less than about 10% by volume of the medium.

The invention further provides a method of culturing stem cells, comprising culturing a stem cell in the medium for the invention.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows the proliferation of hBMSC in the medium for the invention;

FIG. 2 shows the anchorage independent growth of HBMSC in the medium of the invention;

FIG. 3 shows population doubling levels of hBMSC growth in the medium of the invention; and

FIG. 4 shows osteogenic, adipogenic, and chondrogenic differentiation from hBMSCs after induction.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a stem cell medium comprising a fetal bovine serum (FBS), one or more amino acid, one or more vitamin, one or more growth factor, one or more inorganic salt, one or more antioxidant agent, wherein the medium has a calcium concentration of less than 1.8 mM, and the fetal bovine serum is present in an amount of less than about 10% by volume of the medium.

The term “stem cell” is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. The stem cell of the invention includes, but are not limited to, a blood stem cell, an adipose stem cell, a bone marrow mesenchymal stem cell, a mesenchymal stem cell, a neural stem cell, a skin stem cell, an embryonic stem cell, an endothelial stem cell, a hepatic stem cell, a pancreatic stem cell, an intestinal epithelium stem cell, or a germ stem cell.

Generally, high molecular weight protein and unknown growth factor is usually not used in a stem cell medium to avoid the differentiation of the stem cell. Additionally, the source and quality of FBS is unstable. Thus, FBS in the medium of the invention is adjusted below 10% (v/v), preferably, below 5% (v/v), more preferably, about 2%-5% (v/v) to reduce the risk of pathogen contamination.

The stem cell medium of the invention comprises one or more antioxidant agent. For example, vitamin C, vitamin E, N-Acetyl-L-Cysteine (NAC), and/or nicotinamide. In the invention, all possible isomeric forms of vitamin C are included, such as L-ascorbic acid-2-phosphate, L-ascorbate, or ascorbic acid.

In one embodiment, vitamin C has a concentration of less than 0.2 mM, preferably about 0.1-0.2 mM, or less than 0.1 mM. N-Acetyl-L-Cysteine has a concentration of less than 0.5-1.5 mM and nicotinamide has a concentration of less than 0.01-0.02 mg/L.

The stem cell medium of the invention comprises one or more amino acid. The term “amino acid” refers to natural amino acids, unnatural amino acids, and amino acid analogs in their D and L stereoisomers if their structure allow such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine and pipecolic acid. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, S-(carboxymethyl)-cysteine sulfoxide and S-(carboxymethyl)-cysteine sulfone.

The stem cell medium of the invention comprises one or more vitamin. The amino acid includes water soluble vitamin, such as vitamin B group, vitamin C, and/or vitamin H. In one embodiment, vitamin can be a choline chloride, D-pantothenic acid, thiamine, riboflavin, niacinamide, pentothenic acid, pyridoxine, folic acid, biotain, vitamin B-12 and/or vitamin C.

The stem cell medium of the invention comprises one or more inorganic ion salt. The term “inorganic ion salt” refers to any inorganic salt which is suitable for use in a solid electrolyte. The inorganic ion salt preferably contains at least one atom selected from the group consisting of Li, Na, K, Cs, Ag, Cu and Mg. The particular inorganic ion salt employed is not critical and examples of suitable inorganic ion salts include, by way of, example, LiClO₄, LiCl, LiSCN, LiBF₄, LiAsF₆, LiCF₃SO₃, LiPF₆, NaSCN, CsSCN, FeSO₄, CuSO₄, MgCl, AgNO₃, MgSO₄, CuCl₂, MnSO₄, (NH₄)₂MO₄, Na₂HPO₄, NaCl, Na₂SeO₃, NaSiO₃, KH₂PO₄, SnCl₂, ZnSO₄, NiCl₂, KCl, Mg(ClO₄)₂, acetate, adenine and the like. In one embodiment, the inorganic ion salt can be a hydrate, such as CuSO₄.5H₂O, FeSO₄.7H₂O, MgCl.6H₂O, (NH₄)₂MO₄.4H₂O, NiCl₂.6H₂O, NaSiO₃.9H₂O, Na₂HPO₄, SnCl₂.2H₂O and/or ZnSO₄.7H₂O, etc.

It should be noted that stem cells may be stimulated to differentiation at a high calcium concentration condition. Thus, the calcium concentration of the medium for the invention is adjusted below 1.8 mM, preferably, below about 1-1.0 mM, more preferably, 0.8-0.9 mM.

The stem cell medium of the invention comprises one or more growth factor. The term “growth factor” as used herein is a molecule(s) which confers a growth advantage or disadvantage upon a host cell or upon a replicator. Typical growth factors include nutrients, enzymes necessary for metabolism of nutrients, and binding and structural proteins, and proteins involved in replication, metabolism, formation and maintenance of essential structural components, or cellular and subcellular growth. The growth factor includes, but are not limited to, epidermal growth factor, vascular endothelial growth factor, transforming growth factor, nerve growth factor, platelet-derived growth factor, insulin, insulin-like growth factors, glial growth factor, basic fibroblast growth factor, growth hormone, bovine pituitary extract, transferring, recombinant epidermal growth factor, hydrocortisone, triiodothyronine, and/or thymidine.

In one embodiment, epidermal growth factor, basic fibroblast growth factor or/and insulin is an essential ingredients in the stem cell medium of the invention, since they maintain that the stem cells will keep at steady stage for its self-renewal, and provide the growth factors in a low-FBS medium.

The stem cell medium of the invention further comprises HEPES, a hypoxanthine, a linoleic acid, a phenol red, a putrescine, a pyruvic acid, a thioctic acid, and/or a thymidine.

In one embodiment, the stem cell medium of the invention includes FBS, N-Acetyl-L-Cysteine, nicotinamide, EGF, bEGF, insulin, BPE, transferring, rEGF, hydrocortisone, triiodothyronine, thymidine, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and/or valine, choline chloride, D-calcium pantothenate, D-Pantothenic acid, thiamine, riboflavin, niacinamide, pentothenic acid, pyridoxine, folic acid, biotin, vitamin B12, CuSO₄, FeSO₄, MgCl, MgSO₄, MnSO₄, (NH₄)₂MO4, NiCl₂, KCl, KH₂PO₄, acetate, NaCl, NaSiO₃, Na₂HPO₄, Na₂SeO₃, SnCl₂, ZnSO₄, adenine, HEPES, Hypoxanthine, Linoleic Acid, Phenol Red, putrescine, pyruvic Acid, thioctic Acid, and thymidine.

In another embodiment, the stem cell medium of the invention includes FBS, N-Acetyl-L-Cysteine, nicotinamide, EGF, bEGF, insulin, BPE, transferring, rEGF, hydrocortisone, triiodothyronine, thymidine, L-alanine, L-arginine, L-asparagine, L-aspartic acid, L-cysteine, L-methionine, L-glutamine, L-glutamic acid, glycine; L-histidine.HCl.H₂O, L-histidine, L-isoleucine, L-leucine, L-Lysine.HCl, L-methionine, L-phenylalanine, L-proline, L-serine, L-threonine, L-tryptophan, L-tyrosine, L-valine, choline chloride, D-calcium pantothenate, D-Pantothenic acid, thiamine, riboflavin, niacinamide, pentothenic acid, pyridoxine, folic acid, biotin, vitamin B12, CuSO₄.5H₂O, FeSO₄.7H₂O, MgCl.6H₂O, MgSO₄, MnSO₄, (NH₄)₂MO₄.4H₂O, NiCl₂.6H₂O, KCl, KH₂PO₄, Na.acetate, NaCl, NaSiO₃.9H₂O, Na₂HPO₄, Na₂SeO₃, SnCl₂.2H₂O, ZnSO₄.7H2O, adenine.HCl, HEPES, hypoxanthine, Linoleic Acid, phenol red, putrescine, pyruvic acid, thioctic Acid, and thymidine.

The medium of the invention not only maintains the capability of the stem cell, but also avoids the apoptosis or differentiation of the stem cell. Additionally, a large amount of stem cell can be developed soon after the culture begins (about 60 days), and the cell population doubling time (CPDT) of each cell lines is similar.

The stem cell can be cultured to at least the 19th generation in the medium of the invention, and its characteristic does not changed, such as cell population doubling time, cell morphology, cell surface antigen, OCT4 gene expression, etc.

Moreover, the medium of the invention does not affect the differentiation of the stem cells, so that the cultured stem cell still can be differentiated by a proper induction. For example, bone marrow stroma stem cell can be differentiated to bone, fat, cartilage and muscle tissue by ostio-, adipo-, and chondro-induction medium, respectively.

The invention further provides a stem cell culture method comprising cultivating a stem cell in the medium of the invention. The cell culture method of the invention can simply generate stem cell and maintain its properties.

### EXAMPLE

### Example 1

### Isolation of Human Bone Marrow Stroma Cells

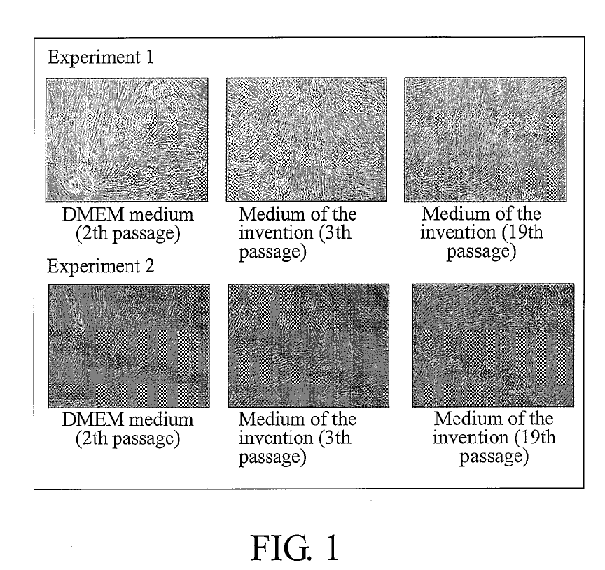

Human bone marrow stroma cells (hBMSCs) were isolated from volunteer patients with 3 hip osteonecrosis, 3 dysplastic osteoarthritis and 4 normal cases. 5 ml of bone marrow was aspirated from ilium crest, and then the nucleated stroma cells were separated by a Percoll™ (Amersham Pharmacia, Piscataway, N.J.) gradient and collected for primary cell cultures (J Bone Joint Surg Am 75, 92-105, 1993). The cells were maintained in Dulbecco modified Eagle medium (GibcoBRL, Gaithersburg, Md.) containing 10 percent fetal bovine serum (Hyclone Laboratories, Logan, Utah), fifty milligrams of sodium ascorbate per milliliter, and antibiotics (100 units of penicillin G per milliliter and 100 micrograms of streptomycin per milliliter) in a humidified atmosphere of 5 percent carbon dioxide at 37° C., and change media every second day. After 15 days, when HBMSCs were attached and about 50% confluence, they were sub-cultured and seeded on the medium of the invention, which was called “passage 2 (P2)”. The passage was as to analogize. The medium of the invention was the modification of MCDB 153 media (Keratinocyte-SFM, GIBCO-Invitrogen Corporation) with 0.95 mM of calcium, 1 mM of N-acetyl-L-cysteine (Sigma A8199), 0.1 mM of L-ascorbic acid 2-phosphate (Sigma A8960), 5 ng/ml of recombinant epidermal growth factor (rEGF), 25 μg/ml of bovine pituitary extract (BPE), 5 μg/ml of insulin, 74 ng/ml of hydrocortisone, and L-cysteine. For the control group, the hBMSCs was incubated in DMEM medium. Referring to FIG. 1, compared with the control group, the serpiginous-shaped hBMSCs appeared in the medium of the invention.

### Example 2

### Analysis of Anchorage Independent Growth

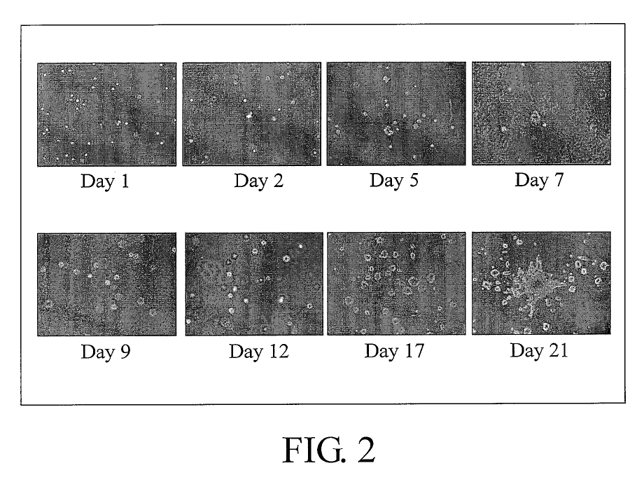

A total of 50,000 hBMSCs in 3 ml of 0.33% agarose medium were plated on top of 3-ml of prehardened 0.5% agarosemedium in each of triplicate dishes (6 cm). Then, 2.5 ml of liquid medium of the invention was added and changed media every 2 days. At the end of 21 days, the numbers of colonies were scored under a microscope with the dish containing anchorage independent growth (AIG) colonies on top of a dish with grids. Referring to FIG. 2, hBMSCs were developed in the soft agar, and 62-65% of hBMSCs displayed anchorage independent growth in the medium of the invention.

### Example 3

### Analysis of Cumulative Population Doubling Level

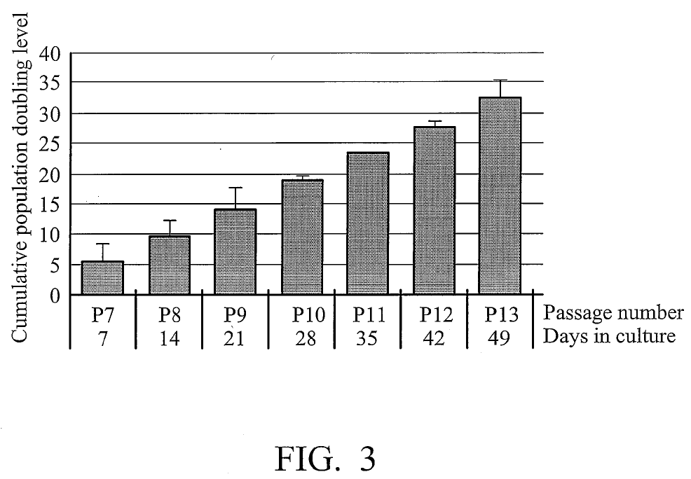

Primary cell culture of hBMSCs were isolated and cultured, which was called P1. When hBMSCs were grown in culture dish until 80% confluence, they were sub-cultured and seeded to a new dish as passage 2 (P2). The passage was as to analogize. Cumulative population doubling level (CPDL) in continual subculture and growth from a known number of cells was calculated to determine the proliferation potential of putative hBMSCs. The CPDL at each subcultivation was calculated from the cell count by using the equation: ln (Nf/Ni)/ln2, where Ni and Nf are initial and final cell numbers, respectively, and ln is the natural log (ln). Referring to FIG. 3, the population doubling time of each cell lines was similar to each other. The population doubling time was about 5 to 10 hours. The data indicated that the medium of the invention did not affect the growth rate of the stem cell.

### Example 4

### Multilineage Differentiation (Adipogenesis, Chondrogenesis, Osteogenesis, and Myogenesis)

For differentiation induction of putative hBMSCs into adipocytes, osteoblasts, and chondrocytes, the cell initially developed and propagated in the media of the invention with 5% FBS, and then were treated by different supplementations in a modified Eagle's medium (Cell 13, 4279-95, 2002; Science 284, 143-7, 1999; Somatic Cell Genet 7, 235-53, 1981).

To induce osteogenic differentiation, 5th to 19th passage cells were treated with osteogenic-induction medium for 12 days, and the induction medium was changed every second day. Osteogenesis was assessed at weekly intervals. Osteogenic medium consists of IMDM supplemented with 0.01 μM of dexamethasone (Sigma-Aldrich, St Louis, Mo.), 50 μM of β-glycerol phosphate (Sigma-Aldrich), and 0.2 mM of L-ascorbic-2-phosphate (Sigma-Aldrich) (Blood 103, 1669-75, 2004; Stem Cells Dev 14, 92-102, 2005).

To induce adipogenic differentiation, 5th to 19th-passage cells were treated with adipogenic-induction medium for 12 days. The induction medium was changed every second day and adipogenesis was assessed at weekly intervals. Adipogenic-induction medium contains DMEM supplemented with 0.5 mM 3-isobutyl-1-methylxanthine (IBMX; Sigma-Aldrich), 1 μM Dexamethasone (Sigma-Aldrich) and 10 μg/ml Insulin. Myogenesis were treated with 10% hydrocortisone 50 μM with 5% horse serum medium changed every second day and chondrogenesis was assessed at weekly intervals. Examination was conducted for myosin gene expression by immunostaining after 4-6 weeks of incubation (Stem Cells Dev 14, 92-102, 2005).

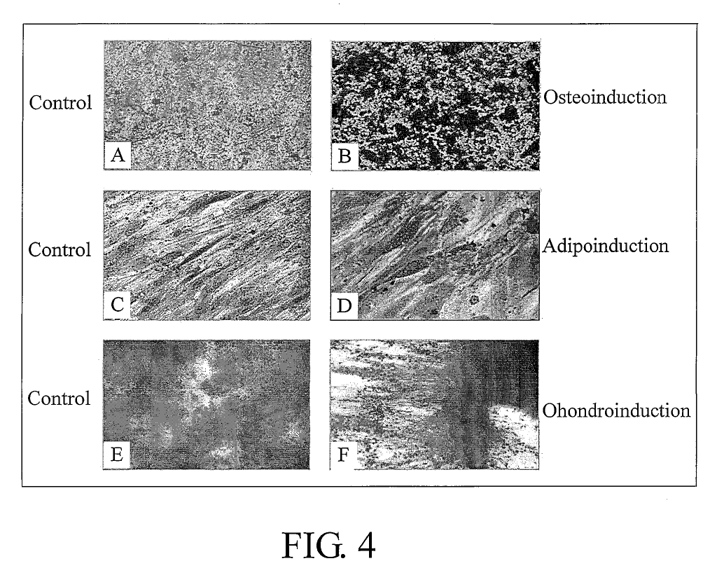

To induce chondrogenic differentiation, 5th to 19th passage cells were transferred into a 15-mL polypropylene tube and centrifuged at 1000 rpm for 5 minutes, and then the supernatant was removed. One million cells into 10 μl media were loaded into a dish and treated with chondrogenic-induction medium for 12 days. The induction medium was changed every second day and chondrogenesis was assessed at weekly intervals. Chondrogenic-induction medium consists of low-glucose DMEM (Gibco, Carlsbad, Calif.) supplemented with 10 ng/ml of TGF-β1 (Sigma-Aldrich), 50 μM of L-Ascorbate β-2-phosphate (Sigma-Aldrich), and 6.25 μg/ml of insulin (Sigma-Aldrich). Referring to FIG. 4, hBMSCs were differentiated into osteoblasts (B), adipocytes (D), and chondrocytes (F) after proper induction.

### Example 5

### Analysis of Cell Surface Marker

Cells were analyzed by FAC scan argon laser cytometer (BD, Biosciences, San Jose, Calif.) using various antibodies. Suitable processes for analyzing cell surface marker include, for example, those illustrated in references such Tissue Eng 7, 211-28, 2001. hBMSCs were cultured in control medium for 72 hours before analysis, and 5×10⁵ cells were incubated with primary antibodies. Firstly, hBMSCs were harvested in 0.25% trypsin/EDTA and then fixed for 30 min in ice-cold 70% EtOH. The fixed cells were washed in flow cytometry buffer (PBS, 2% FBS, 0.2% Tween 20) and incubated for 30 min in flow cytometry buffer containing fluorescein isothiocyanate-conjugated monoclonal antibodies CD antigens: CD29, CD31, CD34, CD44, CD45, CD49d, CD56, CD62e, CD90, CD105, CD106, CD133 and CD166 to determine specific stem cell surface markers. hBMSCs were stained with a phycoerythrin-conjugated nonspecific IgG to assess background fluorescence (Mol Biol Cell 13, 4279-95, 2002). Cell surface marker expression was determined by comparison with isotype control on a histogram plot. The experiment results are listed as in Table 1. According to Table 1, the medium of the invention does not affect or change the stem cell surface markers.

TABLE 1
surface antigen marker

| | | | | hBMSC cultured in |
|---|---|---|---|---|
| | hADSC | hBMSC | Osteoblast-like cells | DMEM medium (P6) |
| CD29 | + | + | + | + |
| CD31 | − | − | + | − |
| CD34 | +/− | − | + | − |
| CD44 | + | + | | |
| CD45 | − | − | − | − |
| CD49d | + | − | + | +/− |
| CD56 | − | − | + | +/− |
| CD62e | − | − | + | +/− |
| CD90 | + | + | | + |
| CD105 | + | + | | + |
| CD106 | − | − | − | − |
| CD133 | − | − | | +/− |
| CD166 | + | + | + | +/− |

| | hBMSC cultured in | hBMSC cultured in | hBMSC cultured in |
|---|---|---|---|
| | the medium of | the medium of | the medium of |
| | the invention (P6) | the invention (P13) | the invention (P15) |
| CD29 | + | + | + |
| CD31 | − | − | − |
| D34 | − | − | − |
| CD44 | | + | + |
| CD45 | − | − | − |
| CD49d | + | + | + |
| CD56 | − | − | − |
| CD62e | − | − | − |
| CD90 | + | + | + |
| CD105 | + | + | + |
| CD106 | − | − | − |
| CD133 | +/− | − | − |
| CD166 | +/− | + | + |

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A medium of culturing a stem cell, comprising a fetal bovine serum, one or more amino acid, one or more vitamin, one or more growth factor, one or more inorganic ion salt, one or more antioxidant agent, wherein the medium has a calcium concentration of less than 1.8 mM, and the fetal bovine serum is present in an amount of less than about 10% by volume of the medium.

2. The medium as claimed in claim 1, wherein the fetal bovine serum is present in an amount from about 2% to about 5% by volume of the medium.

3. The medium as claimed in claim 1, wherein the medium has a calcium concentration of 0.8-0.9 mM.

4. The medium as claimed in claim 1, further comprising a hypoxanthine, a linoleic acid, a phenol red, a putrescine, a pyruvic acid, a thioctic acid, and/or a thymidine.

5. The medium as claimed in claim 1, wherein the antioxidant agent comprises vitamin C, N-acetyl-L-cysteine, or nicotinamide.

6. The medium as claimed in claim 5, wherein the vitamin C is L-ascorbic acid-2-phosphate.

7. The medium as claimed in claim 6, wherein the L-ascorbic acid-2-phosphate is present in a concentration from about 0.1 to 0.2 mM.

8. The medium as claimed in claim 5, wherein the N-acetyl-L-cysteine is present in a concentration from about 0.8 to 1.2 mM.

9. The medium as claimed in claim 5, wherein the nicotinamide is present in a concentration from about 2.0 to 2.8 mM.

10. The medium as claimed in claim 1, wherein the vitamin is a water soluble vitamin.

11. The medium as claimed in claim 1, wherein the vitamin comprises vitamin B group, vitamin C and/or vitamin H.

12. The medium as claimed in claim 1, wherein the vitamin comprises choline chloride, D-calcium pantothenate, D-pantothenic acid, thiamine, riboflavin, niacinamide, pentothenic acid, pyridoxine, folic acid, biotain, vitamin B-12 and/or vitamin C.

13. The medium as claimed in claim 1, wherein the growth factor comprises epidermal growth factor, vascular endothelial growth factor, transforming growth factor, nerve growth factor, platelet-derived growth factor, insulin, insulin-like growth factors, glial growth factor, basic fibroblast growth factor, growth hormone, bovine pituitary extract, transferring, recombinant epidermal growth factor, hydrocortisone, triiodothyronine, and/or thymidine.

14. The medium as claimed in claim 1, wherein the medium has a pH value of about 7.15-7.4.

15. The medium as claimed in claim 1, wherein the inorganic ion salt comprises Li, Na, K, Cs, Ag, Cu and Mg.

16. The medium as claimed in claim 1, wherein the inorganic ion salt comprises LiClO₄, LiCl, LiSCN, LiBF₄, LiAsF₆, LiCF₃SO₃, LiPF₆, NaCl, NaSCN, CsSCN, FeSO₄, CuSO₄, MgCl, AgNO₃, MgSO₄, CuCl₂, MnSO₄, (NH₄)₂MO₄, Na2HPO₄, Na₂SeO₃, NaSiO₃, KH₂PO₄, SnCl₂, ZnSO₄, NiCl₂, KCl, Mg(ClO₄)₂, acetate, adenine, and the like thereof.

17. The medium as claimed in claim 1, wherein the inorganic salt is a hydrate.

18. The medium as claimed in claim 1, wherein the amino acid comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and/or valine.

19. The medium as claimed in claim 1, wherein the stem cell is a bone marrow stroma cell.

20. A method of culturing a stem cell, comprising culturing a stem cell in the medium as claimed in claim 1.
